(19) United States
(12) Patent Application Publication
(10) Pub. No.: US 2008/0131879 A1
(43) Pub. Date: Jun. 5, 2008

(54) Detection of Subjects at Risk For Chd By a Genotype Evaluation Associated With Serum Plant Sterols Enabling Individualized (Drug) Treatment on Demand

(75) Inventors: Jogchum Plat, Maastricht (NL); Ronald P. Mensink, Maastricht (NL)

Correspondence Address: KALOW & SPRINGUT LLP, 488 MADISON AVENUE, 19TH FLOOR, NEW YORK, NY, 10022, US

(73) Assignee: University Van Maastricht, MD Maastricht (NL)

(21) Appl. No.: 11/597,843

(22) PCT Filed: May 30, 2005
(86) PCT No.: PCT/EP05/05851

§ 371 Date: Sep. 18, 2007

(30) Foreign Application Priority Data
May 28, 2004 (EP) 04076589.3

(51) Int. Cl. C12Q 1/68

(52) U.S. Cl. 435/6

(57) ABSTRACT

The present invention provides the use of at least one genotype selected from the group consisting of ABCG8 T400K an ABCG8 D19H as a diagnostic tool for the detection of an increased risk for coronary events in all individual based oil all elevated sitosterol concentration in a blood sample from said individual.

### FIELD OF THE INVENTION

This invention is in the field of coronary heart disease (“CHD”) risk detection and provides certain nucleic acid sequences encoding sterol transporters ABCG8 to identify subjects that are responsive for plant sterol modulating interventions, creating the possibility for individualized (drug) treatment.

### BACKGROUND ART

Plant sterols have been postulated, like cholesterol, as a risk factor for coronary heart disease. The two most abundant plant sterols in nature are campesterol and sitosterol, which are both chemically closely related to cholesterol. Despite the fact that Western diets provide approximately the same amounts of plant sterols (160-360 mg/day) and cholesterol (300 mg/day), plasma concentrations of cholesterol are much higher. This is partly due to the very low absorption of plant sterols, which amounts for campesterol and sitosterol to 1.89 and 0.51%, respectively (1). In contrast, cholesterol absorption varies between 30 and 80% (2).

Two recently discovered ATP binding cassette (ABC) transporters—ABCG5 and ABCG8—play an important role in the regulation of intestinal plant sterol absorption by secreting already absorbed plant sterols out of the enterocytes back into the intestinal lumen (3). ABCG5 and G8 are half-transporters that function together as a heterodimer. Formation of a heterodimer is an absolute necessity to direct the ABCG5/G8 heterodimer from the endoplasmatic reticulum (ER) to the apical membrane (4). This feature explains earlier observations that mutations in only one of the half transporters already causes the rare inheritable autosomal recessive disease sitosterolemia (5-7).

Sitosterolemic patients are characterized by severely elevated serum plant sterol concentrations, normal to moderately increased serum cholesterol concentrations and a high risk to develop coronary heart disease already at a very young age (8, 9). Polymorphisms in the genes encoding ABCG5 and ABCG8 may therefore be related to differences in plant sterol metabolism between subjects. Although not generally accepted, several studies have suggested that elevated concentrations of plant sterols are a risk factor for premature atherosclerosis in sitosterolemic patients (10-12) and even in non-sitosterolemic subjects (13, 14).

Besides the various rare mutations in ABCG5 or ABCG8 as observed in sitosterolemic patients (15), more common sequence variations in both half-transporters—without the sitosterolemic phenotype—have been described. These polymorphisms in ABCG5 and ABCG8 are related to serum plant sterol concentrations (16).

There is, therefore, a need to further investigate whether the above-mentioned genetic variations are also associated to variations in serum plant sterol concentrations during interventions known to affect serum plant sterol metabolism, such as, for example, plant stanol esters which are known for their plant sterol-lowering properties.

### SUMMARY OF THE INVENTION

In accordance with the present invention at least one of the genotypes selected from the group consisting of ABCG8 T400K and ABCG8 D19H is provided as a diagnostic tool for the detection of individuals at risk for CHD by a genotype evaluation based on an elevated sitosterol concentration in blood samples of said individuals.

These and other aspects of the present invention will be described hereinafter in more detail.

### BRIEF DESCRIPTION OF THE DRAWINGS

FIG. 1: Cholesterol standardized campesterol (left), sitosterol (middle), and LDL cholesterol (right) concentrations at the end of the run-in period (upper panels) and the change during the stanol ester feeding period (lower panels) in the different genotypes of the ABCG8 T400K polymorphism. Sitosterol and campesterol concentrations are expressed in 102×μmol/mmol cholesterol, and those of LDL cholesterol per mmol/L. Values are mean ±SE. ^{a}P<0.017, ^{b}P<0.05, ^{c}P=0.053, ^{d}P=0.063.

### DESCRIPTION OF PREFERRED EMBODIMENTS

ABCG5 and ABCG8 play an important role in regulating intestinal plant sterol absorption, and therefore polymorphisms in these genes may be related to differences in plant sterol metabolism between subjects.

Surprisingly, it has now been found that in a number of 112 non-hypercholesterolemic healthy subjects cross-sectional cholesterol-standardized serum campesterol and sitosterol concentrations were significantly associated with ABCG8 T400K genotype. In addition, interactions between the ABCG8 T400K genotype with changes in serum plant sterol concentrations were shown after plant stanol consumption, which lower plant sterol levels. However, no significant associations with serum cholesterol concentrations, neither cross-sectional nor after plant stanol intervention, were found.

For the first time interactions between the ABCG8 T400K genotype with changes in serum plant sterol concentrations were evaluated after a daily consumption of 3.8-4.0 gram plant stanols, which lower plant sterol levels. The reduction of −57.1±38.3 10² μmol/mmol cholesterol for sitosterol in TT subjects was significantly greater as compared to that of −36.0±18.7 in subjects with the TK genotype (P=0.021; 95% CI −39.1 to −3.1 10²×μmol/mmol cholesterol) and to that of −16.9±13.0 in subjects with the KK genotype (P=0.047; 95% CI −85.2 to 4.8 10²×μmol/mmol cholesterol).

Therefore, genetic variation in ABCG8 not only explains cross-sectional differences in serum plant sterol concentrations, but also determines a subjects' responsiveness to changes in serum plant sterols during interventions known to affect plant sterol metabolism. These transporters are not related to changes in serum cholesterol concentrations suggesting that they are primarily involved in plant sterol metabolism and not in cholesterol metabolism.

Serum plant sterol concentrations vary widely between individuals (14, 25), but 16 are within subjects rather stable over time (16, 26). This suggests a strong effect of genetic background on plant sterol metabolism. Indeed, heritability may account for more than 80% of the variability in serum plant sterol concentrations between individuals (16).

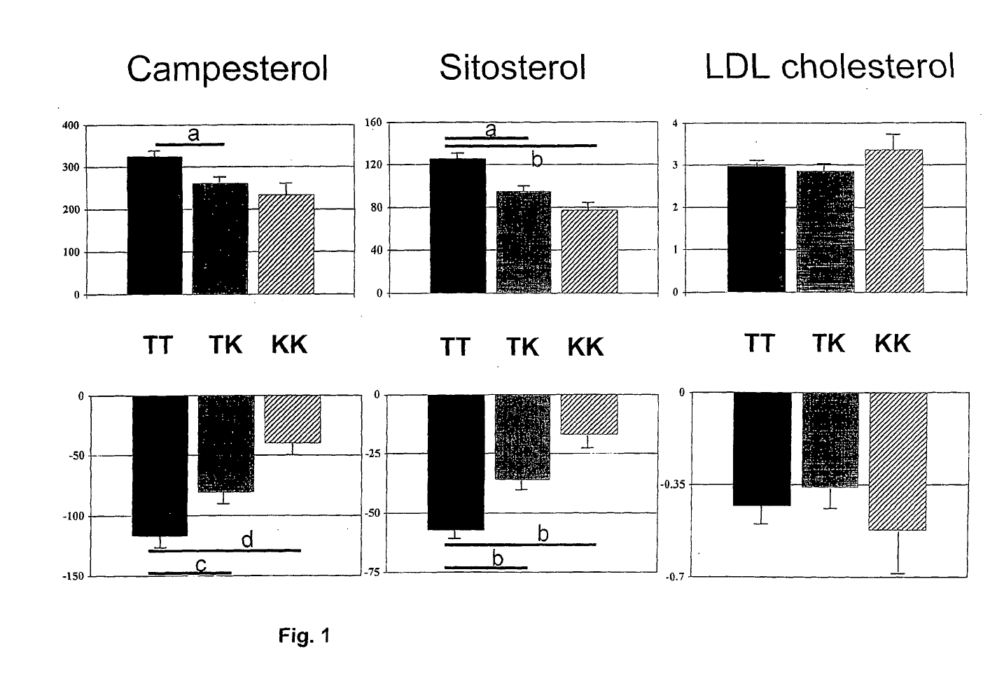

It has now been found in accordance with the present invention that genetic variation in ABCG5 and ABCG8 not only explains cross-sectional serum plant sterol concentrations, but in addition that subjects with high plant sterol concentrations (ABCG8 exon 8, TT genotype) had the largest reduction in serum plant sterol concentrations during plant stanol ester treatment. In particular ABCG8 T400K showed an allele dose dependent relation. See also FIG. 1. Besides for ABCG8 T400K, a similar cross-sectional relation has been described for ABCG8 D19H (16).

These findings indicate that the functionality of the ABCG5/G8 heterodimer is reduced in subjects with the ABCG8 TT genotype. Although the inventors do not wish to be bound to any theory, it is believed that this may result in a lowered transport of plant sterols out of the enterocytes back into the intestinal lumen or from the hepatocytes into bile, and ultimately in increased serum plant sterol concentrations. Why these subjects also showed the largest decrease in serum plant sterol concentrations after consumption of plant stanols is more difficult to explain. It is known that serum plant sterol concentrations are lowered by consumption of plant stanol esters (27-29), probably since plant stanols compete with sterols for incorporation into mixed micelles and hence for uptake into the enterocytes. As plant sterol uptake is not mediated by ABCG5/G8, the lower flux of plant sterols into the enterocyte during plant stanol ester consumption does not depend on ABCG8 genotype. However, if ABCG8 transports the same proportion of plant sterols out of the enterocytes and hepatocytes into the lumen and bile respectively, as before plant stanol ester consumption, it can be calculated that the circulating concentrations of plant sterols in subjects with the TT genotype (with a lower ABCG8 activity) may decrease to a greater degree than in other subjects.

Concerning the potential functionality of the different genotypes it should be remarked that the polymorphic sites of the ABCG8 T400K and D19H polymorphisms are located in a coding region, but are predicted not to contain a transmembrane domain, a signature, or a walker motif (15).

As none of the polymorphisms showed any relationship with (changes in) serum lipid or lipoprotein concentrations, the data according to the present invention do suggest that ABCG8 and ABCG5 do not determine the serum lipoprotein profile. This agrees with observations in mice that disruption of the ABCG8 and ABCG5 genes increased the fractional absorption of dietary plant sterols, but not of cholesterol (3, 18, 19). As a result, serum and hepatic plant sterol concentrations were dramatically increased in ABCG5(−/−)/G8 (−/−) (3, 18) and ABCG5 (−/−) (19) mice, whereas serum cholesterol concentrations were not changed. Cholesterol concentrations in the liver were however increased, which was due to a disturbed biliary cholesterol secretion. Unfortunately, biliary plant sterol secretion was not measured in that study, but in view of the increased hepatic plant sterols levels, it is likely that biliary plant sterol secretion was disturbed as well. In line with these findings, Yu et al (17) reported that over-expression of ABCG8 and ABCG5 significantly lowered serum plant sterol concentrations, whereas serum cholesterol concentrations were again not different between transgenic and wild-type mice. In contrast to what could be expected from the studies with the ABCG5 (−/−)/G8 (−/−) mice, it was measured that these transgenic mice showed a 50% reduction in dietary cholesterol absorption. As discussed however by the authors, measurements of dietary cholesterol absorption were not accurate due to an increase in the intestinal cholesterol pool by the large amount of biliary cholesterol entering the intestinal lumen. In addition, biliary cholesterol levels increased five to seven-fold, while hepatic cholesterol concentration remained unchanged. The unchanged hepatic and serum cholesterol concentrations could be explained by a counteractive two to four-fold increase in hepatic cholesterol synthesis.

Therefore, results in ABCG5/G8 knock-out and over-expressing mice showed that ABCG5 and G8 affects serum plant sterol concentrations, but not those of cholesterol. In addition it seems that ABCG8 and ABCG5 play a role in the intestinal absorption of plant sterols, whereas hepatic ABCG8 and ABCG5 are involved in excretion of cholesterol into bile and possibly also of plant sterols.

Weggemans et al (30) found that subjects with the ABCG5 Q604E EE genotype had higher serum cholesterol concentrations than carriers with the Q allele. Responses to dietary treatments were however not related to this genotype. Other studies however did not observe any associations between these polymorphisms with serum lipid concentrations (16, 31), while we found a significantly higher serum LDL cholesterol concentration in subjects with the QQ genotype as compared to carriers of the E allele. In a recent study, a statistically significant association between the ABCG8 D19H polymorphism and the proportional reduction in LDL cholesterol during atorvastatin treatment was observed (31). As statin treatment may increase plasma plant sterol concentrations (10-12), it would have been interesting to know if changes in serum plant sterol concentrations were also related to this polymorphism in the ABCG8 gene. In relation to this, Gylling and coworkers (33) recently showed that the higher efficacy of atorvaststin treatment in DH/HH subjects might be related to a higher endogenous cholesterol synthesis and a lower cholesterol absorption as compared to DD subjects. Taken together, these studies have not revealed a consistent relationship between ABCG5 or ABCG8 genotypes with serum lipid or lipoprotein concentrations.

The data according to the present invention indicate that ABCG8 genotype can predict to what extent serum plant sterol concentrations change after interventions. Plasma plant sterol concentrations were found to decrease, though increases in cholesterol-standardized serum plant sterol concentrations are possible after treatment with statins (10) and consumption of functional foods enriched with plant sterol esters (27, 32).

Whether these increases also relate to ABCG8 genotype may be relevant to know. In the Scandinavian Simvastatin Survival Study (4S) study, for example, it was found that changes in plasma plant sterol concentrations after 5-year treatment were positively related to baseline values. More importantly, however, recurrence of coronary events during simvastatin treatment was not reduced in subjects from the highest quartile of plasma sterol concentrations at baseline, despite the fact that reductions in serum total cholesterol concentrations were comparable in all quartiles (10, 12). These observations do of course not prove that increased plant sterol concentrations counteract the beneficial effects of statins on cardiovascular risk.

In conclusion, serum cholesterol-standardized campesterol and sitosterol concentrations, as well as their changes after consumption of plant stanol enriched foods, are related to a variation in the ABCG8 gene, which is present in about 70% of the population. No relations with serum lipid and lipoprotein concentrations were observed. This suggests that changes in the functionality of the ABCG5/G8 heterodimer, mainly affects plasma sterol concentrations, but not those of cholesterol. Whether these findings should have consequences for a patients' optimal cholesterol-lowering drug treatment, warrants further investigation.

Evaluation of ABCG8 and ABCG5 nucleic acid sequences, such as for example the ABCG8 T400K and D19H polymorphisms can be used as a biomarker for the detection of future coronary events. This predictive characteristic originates from its association with cholesterol-standardized serum plant sterol concentrations. In particular, the genotypes ABCG8 T400K (“TT”) and ABCG8 D19H (“DD”) show a strong cross-sectional association with cholesterol-standardized serum plant sterol concentrations and in addition TT subjects show the largest responsiveness towards plant sterol modulating interventions. This enlarged responsiveness has not yet been shown for DD subjects but is likely to be present. Therefore, knowledge regarding a subjects ABCG8 T400K and probably also someone's ABCG8 D19H genotype can be used to predict which subjects will respond with a large increase in serum plant sterols upon treatment using strategies known to elevate cholesterol-standardized serum plant sterol concentrations.

An example of such a therapeutic strategy is the treatment of hyper-cholesterolemic patients with HMGCoA reductase inhibitors (i.e. statins), plant sterol enriched functional foods, or a combination of both. Before starting these types of treatments it would be of great importance to determine a subject's ABCG8 T400K or D19H genotype since in particular TT and DD subjects may respond with a strong increase in cholesterol-standardized serum plant sterol concentrations. Alternatively these patients should be treated with serum cholesterol lowering drugs or other interventions from which it is known that they do not elevate serum plant sterol concentrations. Therefore evaluation of a subjects ABCG8 and ABCG5 genetic make up can be used as a diagnostic tool in the prediction of future coronary events, particularly when optimal drug treatment is considered.

Ultimate prove concerning the true predictive value of these new biomarkers can be obtained from large prospective intervention trials in which a large group of patients are followed for 5 to 10 years (while registrating characteristics of morbidity and mortality) during treatment with placebo or statins (known to elevate cholesterol-standardized serum plant sterol concentrations). After ending the follow-up period ABCG5 and ABCG8 nucleic acid sequences should be determined and related to morbidity and mortality rates in the placebo and statin treated groups. In case there is a clear association between mortality rates and for example ABCG8 T400K TT and/or D19H DD genotype, this is highly indicative that our assumption regarding the predictive value of this genotype is correct. In addition, evaluation of changes in serum plant sterol concentrations during the statin treatment period can be supportive. In the past numerous of these trials have been carried out. One example is the famous 4S trial in which 4444 hypercholesterolemic patients with coronary heart disease were treated with simvastatin for a period of 5.4 years, simvastatin was found to significantly lower morbidity and mortality rates. Relative risk of death in the simvastain group was 0.70 and relative risk of coronary death was 0.58. Although the number of coronary events were lower in the simvastatin group (n=431) as compared to the placebo group (n=622) resulting in a 0.66 relative risk, there were still 431 coronary events and 111 coronary deaths in the simvastatin group. In 2000 regarding the Finnish subcohort of the 4S trial (8868 patients), Miettinen and coworkers (10) reported that the difference between statin treated patients that did not survive 5 years follow up on statin treatment showed a significant higher increase in serum campesterol concentrations as compared to the patients that survived the 5 years follow-up period on statin treatment (Miettinen ATVB 2000). Since the reduction in serum LDL cholesterol was similar in both groups, it was concluded that the elevation in serum campesterol was one of the possible explanations for the observed difference in survival. One of the opportunities to prove the value of our new biomarker will be evaluation of ABCG8 nucleic acid sequences in this 4S cohort and show that the died patients were more often carriers of the TT and/or DD genotype as compared to the surviving patients. Besides the 4S trial, there are many potentially interesting statin trials—with a similar design however another statin—from which evaluation of ABCG8 nucleic acid sequences in relation to mobidity and mortality rates could be confirmative.

Analysis of known ABCG5 and ABCG8 SNPs, for example ABCG8 T400K or D19H, can be carried out, for example, on a Taqman realtime PCR using allelic discrimination methodology and corresponding software as developed by Applied Biosystems. By using this approach we are able to determine for example ABCG8 T400K genotype of approximately 300 patients a day.

The following Examples illustrate the invention and are in no way intended to limit the scope of the invention in any respect.

### EXAMPLES

### Materials and Methods

### Subjects, Diets and Design

Details of the study have been described before (20, 21). In brief, 112 healthy non-hypercholesterolemic volunteers (41 males and 71 females) from Maastricht and surrounding areas were asked to replace during a four-week run-in period their habitual margarines and baking fats for a low erucic acid rapeseed oil (LEAR) based margarine and shortening. For the next eight weeks subjects were randomly allocated, stratified for gender and age, to one of the three intervention groups. The control group (N=42) continued to use the rapeseed-oil based margarine and shortening, while the second and the third group used the same margarine and shortening to which a vegetable-oil based (N=36) or a wood-based (N=34) plant stanol ester mixture was added. The compositions of the experimental products have been described in detail before (22). The margarine was used at breakfast and lunch, and the shortening at diner. During the intervention period, daily intake of plant stanols in the vegetable-oil based group was 3.8±0.6 g (means ±SD) and in the wood-based group 4.0±1.8 g. Plant stanols were esterified with fatty acids from rapeseed oil. All experimental products were prepared by the RAISIO GROUP, Raisio, Finland. Energy intake and the proportions of energy from carbohydrates, fatty acids, and protein, as well as cholesterol and fibre intake did not change during the study (20).

### Blood Sampling and DNA Isolation

At the start of the study (day 1) blood was sampled in a 10 ml EDTA tube (Monoject sterile, Sherwood Medical, Ballymoney, North Ireland), which was used for DNA isolation. At the end of the run-in period (weeks 3 and 4) and at the end of the experimental period (weeks 11 and 12), blood was sampled in a 10 mL clotting tube (CORVAC, integrated serum separator tube, Sherwood Medical Company, St Louis USA) after an overnight fast. Subjects abstained from drinking alcohol the day preceeding, and from smoking on the morning of blood sampling. Serum was prepared by centrifugation at 2000×g for thirty minutes at 4° C., minimally one hour after venipuncture, and aliquots were stored directly at −80° C. for analysis of serum lipids, lipoproteins, plant sterols, plant stanols and cholesterol precursor concentrations at the end of the study (20, 21).

### Analysis of Genetic Variation in ABCG5 and ABCG8

Genotyping of exons 2, 8, and 13 from ABCG8 and of exon 13 from ABCG5 was performed by analysis of restriction fragment length polymorphisms (RFLPs), as described (15). In brief, PCR amplifications were performed in 20 μL volumes containing 350 ng genomic DNA, 25 pmol of each nucleotide primer (Sigma Genosys, Cambridge, UK), 1 unit Taq polymerase (Pharmacia Biotech, Roosendaal, The Netherlands), 0.2 mM of each dNTP (Pharmacia Biotech, Roosendaal, The Netherlands), and 1.5 mM MgCl₂. Before amplification, each sample was denaturated for 5 minutes at 95° C. For ABCG8 genotyping each of the following thirty cycles consisted of 15 seconds at 96° C. (denaturation), 15 seconds at 60° C. (primer annealing), and 30 seconds at 72° C. (extension), followed by 10 minutes at 72° C. (elongation). For ABCG5 genotyping the program was slightly modified in that annealing occurred for 30 seconds at 60° C. and no elongation step was used. PCR products were digested with specific restriction enzymes and the DNA fragments obtained were electrophoresed for 1.5 hours at 125V on a 2.5% agarose gel containing gelstar (Sanvertech, Heerhugowaard, The Netherlands). DNA fragments were visualized by UV at 312 nm using a Wratten gelatine filter on a VDS Imagemaster (Pharmacia Biotech, San Francisco, USA). Sequences of the primers have been described (15). The restriction enzymes (New England Biolabs, Beverly Mass., USA) used were SexAI, MseI, and NcoI for ABCG8 exon 2, 8, and 13 respectively, and XmnI for ABCG5 exon 13.

### Statistics

Due to the limited number of subjects, heterozygous and homozygous carriers of the genetic variants (ABCG8 Y54C; [YC+YY], ABCG8 T400K; [TK+KK], ABCG8 A632V; [VV+VA], ABCG5 Q604E; [QE+EE]) were combined before data analysis. Excluding the homozygous carriers did, however, not affect the conclusions.
At the end of the run-in period cross-sectional differences in metabolic parameters between genotype groups were compared with an unpaired t-test. When significant, the three genotype groups were also compared using ANOVA with Bonferroni correction to examine if relations were dose allele-dependent. In view of their well-known relationships with lipid metabolism, BMI, gender and age were considered as potential confounders. However, these parameters were not significantly different between the various genotype groups (results not shown) and were therefore not included into the models.

In search for gene-diet interactions, changes in metabolic parameters were calculated for each subject as differences between values of the experimental period and run-in period. Since responses in serum lipoprotein, plant sterol and stanol concentrations were not different between the two experimental groups (20, 21) results of the vegetable-oil based and the wood-based plant stanol esters groups were combined (N=70). Effects of genotype on these responses were examined with an unpaired t-test. Again, allele dose-dependent relationships were examined by ANOVA plus Bonferroni correction. All statistical analyses were performed with Statview 4.5 (24).

### Results

Frequency distributions for the genotypes in exons 2 (Y54C), 8 (T400K), and 13 (A632V) of ABCG8 and in exon 13 (Q604E) of ABCG5 are shown in Table 1.

TABLE 1
Frequency distributions of the different genotypes in exons 2
(Y54C), 8 (T400K), and 13 (A632V) of ABCG8, and exon 13
(Q604E) of ABCG5

| All | Control group | Experimental group |
|---|---|---|
| Number of subjects | | |
| 112 | 43 | 70 |

ABCG8 Y54C

| | | | |
|---|---|---|---|
| CC | 18 (16.1) | 6 (14.3) | 12 (17.1) |
| YC | 92 (82.1) | 35 (83.3) | 57 (81.4) |
| YY | 2 (1.8) | 1 (2.4) | 1 (1.4) |

ABCG8 T400K

| | | | |
|---|---|---|---|
| TT | 77 (68.7) | 30 (71.4) | 47 (67.1) |
| TK | 31 (27.7) | 11 (26.2) | 20 (28.6) |
| KK | 4 (3.6) | 1 (2.4) | 3 (4.3) |

ABCG8 A632V

| | | | |
|---|---|---|---|
| VV | 5 (4.5) | 2 (4.8) | 3 (4.3) |
| VA | 37 (33.0) | 17 (40.5) | 20 (28.6) |
| AA | 70 (62.5) | 23 (54.8) | 47 (67.1) |

ABCG5 Q604E

| | | | |
|---|---|---|---|
| QQ | 81 (72.3) | 33 (78.6) | 48 (68.6) |
| QE | 29 (25.9) | 9 (21.4) | 20 (28.6) |
| EE | 2 (1.8) | 0 (0) | 2 (2.9) |

Numbers are absolute frequencies (relative frequencies in parentheses)

Except for ABCG8 Y54C, all genotype frequency distributions were in Hardy-Weinberg equilibrium. The various polymorphisms were not related. At the end of the four-week run-in period, serum concentrations of LDL cholesterol, HDL cholesterol, and triacylglycerol in all subjects (N=112) were 2.95±0.78, 1.59±0.38, and 0.92±0.52 mmol/L, respectively. Serum concentrations of plant sterols, plant stanols, and lathosterol at the end of the run-in period are shown in Table 2.

TABLE 2
Relationships between genetic polymorphisms in ABCG8 and ABCG5
with serum non-cholesterol sterol concentrations.

| | Campesterol | Sitosterol | Lathosterol | Campestanol | Sitostanol |
|---|---|---|---|---|---|
| All | 303.4 ± 98.0 | 115.0 ± 44.5 | 97.3 ± 33.5 | 9.8 ± 6.6 | 7.6 ± 4.3 |

ABCG8 Y54C

| | | | | | |
|---|---|---|---|---|---|
| CC | 368.3 ± 93.3^{a} | 131.3 ± 40.1 | 90.4 ± 22.7 | 12.2 ± 9.5 | 7.0 ± 2.4 |
| YC/YY | 291.0 ± 94.3 | 111.8 ± 44.9 | 98.7 ± 35.2 | 9.3 ± 5.8 | 7.7 ± 4.5 |

ABCG8 T400K

| | | | | | |
|---|---|---|---|---|---|
| TT | 324.2 ± 98.5^{b} | 125.2 ± 45.8^{b} | 93.2 ± 35.0^{c} | 9.6 ± 6.8 | 7.5 ± 4.4 |
| TK/KK | 257.7 ± 80.8 | 92.4 ± 31.9 | 106.4 ± 28.4 | 10.2 ± 6.1 | 7.7 ± 3.9 |

ABCG8 A632V

| | | | | | |
|---|---|---|---|---|---|
| VV/VA | 299.3 ± 103.3 | 111.0 ± 39.8 | 101.0 ± 35.8 | 10.6 ± 7.2 | 7.6 ± 4.4 |
| AA | 305.8 ± 95.3 | 117.3 ± 47.3 | 95.2 ± 32.2 | 9.3 ± 6.1 | 7.6 ± 4.2 |

ABCG5 Q604E

| | | | | | |
|---|---|---|---|---|---|
| QQ | 310.5 ± 98.8 | 118.3 ± 43.7 | 95.3 ± 34.8 | 8.9 ± 5.7 | 7.6 ± 4.3 |
| QE/EE | 284.8 ± 94.9 | 106.2 ± 46.2 | 102.7 ± 29.9 | 11.9 ± 8.1 | 7.6 ± 4.2 |

All values are expressed in 10² × μmol/mmol cholesterol and are means ± SD, and were analyzed after a four-week period consumption of rapeseed oil based margarine and shortening.
^{a}ABCG8 Y54C: CC versus YC/YY subjects (p < 0.01)
^{b}ABCG8 T400K: TT versus TK/KK subjects (p < 0.001)
^{c}ABCG8 T400K: TT versus TK/KK subjects (p = 0.053)

Both ABCG8 Y54C and ABCG8 T400K polymorphisms were significantly related with cholesterol-standardized serum campesterol concentrations, while cholesterol-standardized serum sitosterol concentrations were only associated with ABCG8 T400K genotype (Table 2). The relationship between the ABCG8 T400K polymorphism with cholesterol-standardized serum campesterol or sitosterol concentrations was allele-dependent (FIG. 1). The association with cholesterol-standardized serum lathosterol concentrations nearly reached statistical significance (P=0.053). Differences for cholesterol-standardized serum plant stanol (sitostanol and campestanol) concentrations between the different genotypes never reached statistical significance (Table 2). No associations between the polymorphisms in the ABCG8 gene with serum lipids and lipoproteins were found. Subjects with the QQ genotype (ABCG5 Q604E) showed significantly higher serum LDL cholesterol concentrations as compared with QE/EE subjects (Table 3).

TABLE 3
Relationships between genetic polymorphisms in ABCG8 and ABCG5
with serum lipid and lipoprotein concentrations.

| | LDL-C | HDL-C | TG |
|---|---|---|---|
| All | 2.95 ± 0.78 | 1.59 ± 0.38 | 0.92 ± 0.52 |

ABCG8 Y54C

| | | | |
|---|---|---|---|
| CC | 3.13 ± 0.93 | 1.46 ± 0.30 | 0.78 ± 0.26 |
| YC/YY | 2.91 ± 0.75 | 1.62 ± 0.39 | 0.95 ± 0.55 |

ABCG8 T400K

| | | | |
|---|---|---|---|
| TT | 2.97 ± 0.74 | 1.59 ± 0.37 | 0.85 ± 0.47 |
| TK/KK | 2.89 ± 0.86 | 1.60 ± 0.40 | 1.08 ± 0.58 |

ABCG8 A632V

| | | | |
|---|---|---|---|
| VV/VA | 2.81 ± 0.77 | 1.61 ± 0.36 | 0.93 ± 0.58 |
| AA | 3.03 ± 0.78 | 1.58 ± 0.39 | 0.91 ± 0.48 |

ABCG5 Q604E

| | | | |
|---|---|---|---|
| QQ | 3.04 ± 0.75^{a} | 1.56 ± 0.35 | 0.89 ± 0.45 |
| QE/EE | 2.70 ± 0.81 | 1.68 ± 0.43 | 0.99 ± 0.65 |

All values are expressed in mmol/L and are means ± SD and were analyzed after a four-week period consumption of rapeseed oil based margarine and shortening.
LDL-C: LDL cholesterol,
HDL-C: HDL cholesterol,
TG: triacylglycerol.
^{a}ABCG5 Q604E: QQ versus QE/EE subjects (p < 0.05)

In the 70 subjects that consumed the plant stanol ester enriched margarines, cholesterol-standardized serum campesterol and sitosterol concentrations decreased, while those of lathosterol increased as compared to the control group (Table 4).

TABLE 4
Relationships between genetic polymorphisms in ABCG8
and ABCG5 with changes in serum non-cholesterol sterol
concentrations after consumption of plant stanol esters

| | | | | |
|---|---|---|---|---|
| Campesterol | Sitosterol | Lathosterol | Campestanol | Sitostanol |

Experimental group

| | | | | | |
|---|---|---|---|---|---|
| Control | −5.9 ± 39.2 | −7.8 ± 22.4 | 0.5 ± 19.5 | −0.6 ± 7.2 | 0.4 ± 4.0 |
| Stanol | −103.1 ± 70.6* | −49.3 ± 34.9* | 17.4 ± 22.2* | 6.3 ± 8.1* | 8.2 ± 4.5* |

Genotype group
ABCG8 Y54C

| | | | | | |
|---|---|---|---|---|---|
| CC | −128.9 ± 57.1 | −52.5 ± 30.8 | 12.9 ± 24.5 | 6.2 ± 11.0 | 9.5 ± 4.3 |
| YC/YY | −97.8 ± 72.4 | −48.7 ± 35.9 | 18.3 ± 21.8 | 6.3 ± 7.4 | 7.9 ± 4.5 |

ABCG8 T400K

| | | | | | |
|---|---|---|---|---|---|
| TT | −116.7 ± 76.5^{a} | −57.1 ± 38.3^{b} | 16.5 ± 19.8 | 6.1 ± 8.3 | 8.4 ± 4.5 |
| TK/KK | −75.4 ± 46.9 | −33.5 ± 19.0 | 19.2 ± 26.7 | 6.8 ± 7.6 | 7.7 ± 4.6 |

ABCG8 A632V

| | | | | | |
|---|---|---|---|---|---|
| VV/VA | −94.6 ± 60.3 | −49.4 ± 25.9 | 20.2 ± 18.3 | 6.7 ± 7.8 | 8.0 ± 4.9 |
| AA | −107.3 ± 75.4 | −49.3 ± 38.8 | 16.0 ± 23.9 | 6.1 ± 8.3 | 8.3 ± 4.4 |

ABCG5 Q604E

| | | | | | |
|---|---|---|---|---|---|
| QQ | −106.3 ± 75.6 | −50.9 ± 36.9 | 14.7 ± 21.3 | 6.3 ± 8.2 | 7.6 ± 4.2 |
| QE/EE | −96.1 ± 59.2 | −46.0 ± 30.6 | 23.2 ± 23.5 | 6.3 ± 7.9 | 9.5 ± 4.9 |

All values are expressed in 10² × ∝mol/mmol cholesterol and are means ± SD. Changes in all parameters were calculated as the difference between values at the end of the run-in period (week 3 + 4) and the experimental period (week 11 + 12).
*P < 0.001 control versus stanol group
^{a}ABCG8 T400K, TT versus TK/KK subjects (P < 0.05)
^{b}ABCG8 T400K, TT versus TK/KK subjects (P < 0.01)

These changes indicated that plant stanol ester consumption lowered cholesterol absorption and increased cholesterol synthesis, which is in agreement with the known underlying mechanism. As a consequence LDL cholesterol concentrations were significantly lowered. HDL and triacylglycerol concentrations were not affected (Table 5).

TABLE 5
Relationships between genetic polymorphisms in ABCG8 and ABCG5
with changes in lipid and lipoprotein concentrations after consumption
of plant stanol esters

| | LDL-C | HDL-C | TG |
|---|---|---|---|
| Control | −0.06 ± 0.36 | 0.01 ± 0.16 | 0.02 ± 0.23 |
| Stanols | −0.41 ± 0.31* | 0.01 ± 0.12 | −0.04 ± 0.30 |

ABCG8 Y54C

| | | | |
|---|---|---|---|
| CC | −0.49 ± 0.30 | 0.03 ± 0.10 | 0.01 ± 0.18 |
| YC/YY | −0.40 ± 0.31 | 0.00 ± 0.12 | −0.05 ± 0.32 |

ABCG8 T400K

| | | | |
|---|---|---|---|
| TT | −0.43 ± 0.32 | 0.02 ± 0.11 | −0.01 ± 0.22 |
| TK/KK | −0.38 ± 0.30 | −0.01 ± 0.13 | −0.09 ± 0.42 |

ABCG8 A632V

| | | | |
|---|---|---|---|
| VV/VA | −0.41 ± 0.29 | −0.00 ± 0.13 | −0.10 ± 0.38 |
| AA | −0.42 ± 0.32 | 0.01 ± 0.12 | −0.00 ± 0.25 |

ABCG5 Q604E

| | | | |
|---|---|---|---|
| QQ | −0.44 ± 0.30 | 0.01 ± 0.10 | −0.01 ± 0.34 |
| QE/EE | −0.36 ± 0.34 | −0.01 ± 0.16 | −0.09 ± 0.18 |

All values are means ± SD. Changes in all parameters were calculated as the difference between values at the end of the run-in period (week 3 + 4) and the experimental period (week 11 + 12).
*P < 0.001 control versus stanol group.

Changes in cholesterol-standardized serum campesterol and sitosterol concentrations were significantly associated with the ABCG8 T400K polymorphism (table 4, FIG. 1). The decrease of −116.7±76.5 10²×μmol/mmol cholesterol for campesterol in TT subjects was significantly different from the change of −75.4±46.9 10²×μmol/mmol in subjects with the TK/KK genotype (P<0.05; 95% CI, −76.0 to −6.6 10²×μmol/mmol cholesterol). The reduction of −57.1±38.3 10² μmol/mmol cholesterol for sitosterol in TT subjects was also larger as compared to the reduction of −33.5±19.0 10²×μmol/mmol cholesterol in subjects with the TK/KK genotypes (P<0.01; 95% confidence interval (CI) for the difference in changes, −40.5 to −6.7 10²×μmol/mmol cholesterol). Additional analysis to evaluate the association between the three different genotype groups of the ABCG8 T400K polymorphism (TT, TK, and KK) with changes in serum plant sterol and lipoproteins concentrations, suggested an allele-dependent relation for both cholesterol-standardized serum campesterol and sitosterol concentrations (FIG. 1). The reduction of −57.1±38.3 10² μmol/mmol cholesterol for sitosterol in TT subjects was significantly greater as compared to that of −36.0±18.7 in subjects with the TK genotype (P=0.021; 95% CI −39.1 to −3.1 10²×μmol/mmol cholesterol) and to that of −16.9±13.0 in subjects with the KK genotype (P=0.047; 95% CI −85.2 to −4.8 10²×μmol/mmol cholesterol). Changes in serum campesterol concentrations showed a comparable association pattern with the ABCG8 T400K genotype. No statistically significant relationships between the ABCG8 and ABCG5 polymorphisms with changes in serum plant stanol (table 4), lipids, and lipoproteins (table 5) concentrations were found.

Finally, results indicate that subjects with the genotype associated with high cross-sectional serum plant sterol concentrations and a large reduction in serum plant sterol concentrations upon plant stanol ester intervention (ABCG8 TT and ABCG5 EE) are characterized by a higher increase in cholesterol standardized plant stanol concentrations during plant stanol ester enriched margarine consumption.

### REFERENCES

1. Ostlund R E, McGill J B, Zeng C M, et al. Gastrointestinal absorption and plasma kinetics of soy Delta(5)-phytosterols and phytostanols in humans. Am J Physiol Endoctinol Metab. 2002; 282:E911-6.
2. Bosner M S, Lange L G, Stenson W F, et al. Percent cholesterol absorption in normal women and men quantified with dual stable isotopic tracers and negative ion mass spectrometry. J Lipid Res. 1999; 40:302-8.
3. Yu L., Hammer R E, Li-Hawkins J, et al. Disruption of ABCG5 and ABCG8 in mice reveals their crucial role in biliary cholesterol secretion. PNAS. 2002; 99:16237-16242.
4. Graf G A, Li, W P, Gerard R D, et al. Coexpression of ATP-binding cassette proteins ABCG5 and ABCG8 permits their transport to the apical surface. J Clin Invest. 2002; 1110:659-669.
5. Lee M., Lu K, Patel S. Genetic basis of sitosterolemia. Curr Opin Lipidol. 2001; 12:141-149.
6. Berge K E, Tian H, Graf G A, et al. Accumulation of dietary cholesterol in sitosterolemia caused by mutations in adjacent ABC transporters. Science. 2000; 290:1771-1775.
7. Lee M H, Lu K, Hazard S, et al. Identification of a gene, ABCG5, important in the regulation of dietary cholesterol absorption. Nat Gen. 2001; 27:79-83.
8. Bhattacharyya A K, Connor W. β-sitosterolemia and xanthomatosis. J Clin Invest. 1974; 53:1033-1043.
9. Björkhem I, Boberg K M. Inborn errors in bile acid biosynthesis and storage of sterols other than cholesterol. In The metabolic basis of inherited diseases. C. R. Scriver, A. L. Beaudet, W. S. Sly, and D. Valle, editors. McGraw Hill, New York, N.Y. 1994; 2073-2100.
10. Miettinen T A, Strandberg T E, Gylling H. Noncholesterol sterols and cholesterol lowering by long-term simvastatin treatment in coronary patients; relation to basal serum cholestanol. Arterioscier Thromb Vasc Biol. 2000; 20:1340-1346.
11. Miettinen T A, Gylling H, Lindbohm N, et al. Serum noncholesterol sterols during inhibition of cholesterol synthesis by statins. J Lab Clin Med. 2003; 141:131-137.
12. Miettinen T A, Gylling H, Strandberg T, et al. Baseline serum cholestanol as predictor of recurrent coronary events in subgroup of Scandinavian simvastatin survival trial. BMJ. 1998; 316:1127-1130.
13. Sudhop T, Gottwald B M, Von Bergmann K. Serum plant sterols as a potential riskfactor for coronary heart disease. Metabolism. 2002; 51:1519-1521.
14. Glueck C J, Speirs J, Tracey T, et al. Relationships of serum plant sterols (phytosterols) and cholesterol in 595 hypercholesterolemic subjects, and familial aggregation of phytosterols, cholesterol, and premature coronary heart disease in hypercholesterolemic probands and their first degree relatives. Metabolism. 1991; 40:842-848.
15. Hubacek, J A, Berge K E, Cohen J C, et al. Mutations in ATP-binding cassette binding proteins G5 (ABCG5) and G8 (ABCG8) causing sitosterolemia. Human Mutation. 2001; 452:1-5.
16. Berge, K E, Von Bergmann K, Lutjohann D, et al. Heritability of plasma noncholesterol sterols and relationship to DNA sequence polymorphism in ABCG5 and ABCG8. J Lipid Res. 2002; 43:486-494.
17. Yu L, Li-Hawkins J, Hammer R E, et al. Overexpression of ABCG5 and ABCG8 promotes biliary cholesterol secretion and reduces fractional absorption of dietary cholesterol. J Clin Invest. 2002; 110:671-680.
18. Yu L, York J, Von Bergmann K, et al. Stimulation of cholesterol excretion by the liver X receptor agonist requires ATP-binding Cassette transporters G5 and G8. J Biol Chem. 2003; 278:15565-15570.
19. Plosch T, Bloks V W, Terasawa Y, et al. Sitosterolemia in ABC-transporter G5 deficient mice is aggravated on activation of the liver-X receptor. Gastroenterology 2004; 126:290-230.
20. Plat J. Mensink R. Vegetable oil based versus wood based stanol ester mixtures: effects on serum lipids and hemostatic factord in non-hypercholesterolemic subjects. Atlherosclerosis. 2000; 148:101-112.
21. Plat J, Mensink R P. Effects of diets enriched with two different plant stanol ester mixtures on plasma ubiquinol-10 and fat-soluble antioxidant concentrations Metabolism 2001; 50:520-529.
22. Plat J, Onselen van E N M, Mensink R P. Dietary plant stanol ester mixtures: effects on safety parameters and erythrocyte membrane fatty acid composition in non hypercholesterolemic subjects. Eur Heart J. 1999; 1 (Suppl):s58-s63
23. Plat J, Mensink R. Relationship of genetic variation in genes encoding apolipoprotein A-IV, scavenger receptor BI, HMG-CoA reductase, CETP and apolipoprotein E with cholesterol metabolism and the response to plant stanol ester consumption. Eur J Clin Invest. 2002; 32:242-250.
24. Statview (for the Macintosh) manual. 1992, Statview Abacus Concepts Inc: Berkely.
25. Kempen H J M, Glatz J F C, Gevers Leuven J A, et al. Serum lathosterol concentration is an indicator of whole-body cholesterol synthesis in humans. J Lipid Res. 1988; 29, 1149-1155.
26. Li J H, Awad A B, Fink C S, et al. Measurement variability of plasma beta-sitosterol and campesterol, two new biomarkers for cancer prevention. Eur J Cancer Prev. 2001; 10:245-249.
27. Weststrate J A Meijer G W. Plant sterol-enriched margarines and reduction of plasma total- and LDL-cholesterol concentrations in normocholesterolaemic and mildly hypercholesterolaemic subjects. Eur J Clin Nutr. 1998; 52:334-43.
28. Gylling H, Radhakrishnan R Miettinen T A. Reduction of serum cholesterol in postmenopausal women with previous myocardial infarction and cholesterol malabsorption induced by dietary sitostanol ester margarine: women and dietary sitostanol. Circulation. 1997; 96:4226-31.
29. Miettinen T A, Puska P, Gylling H, et al. Reduction of serum cholesterol with sitostanol-ester margarine in a mildly hypercholesterolemic population. N Engl J. Med. 1995; 333:1308-12.
30. Weggemans, R M, Zock P L, Tai E S, et al. ATP binding casette G5 C1950G polymorphism may affect blood cholesterol concentrations in humans. Clin Genet. 2002; 62:226-229.
31. Kajinami K, Brousseau M E, Nartsupha C, et al. ATP binding cassette transporter G5 and G8 genotypes and plasma lipoprotein levels before and after treatment with atorvastatin. J Lipid Res. 2004; epub ahead.
32. Naumann E, Plat J, Mensink R P. Changes in serum concentrations of non-cholesterol sterols and lipoproteins in healthy subjects do not depend on the ratio of plant stanols in the diet. J. Nutr. 2003; 133:2741-2747.
33. Gylling et al, J Lipid Research 2004; 45:1660-1665)

Each of the above cited publications is herein incorporated by reference to the extent to which it is relied on herein.

1. Use of at least one genotype selected from the group consisting of ABCG8 T400K and ABCG8 D19H as a diagnostic tool for the detection of an increased risk for coronary events in an individual based on an elevated sitosterol concentration in a blood sample from said individual.

2. A method of assessing a risk of death due to coronary disease in a subject comprising:
(a) obtaining a sample from the subject wherein the sample comprises nucleic acids from the subject;
(b) determining from the sample at least one genotype selected from the group consisting of ABCG8 T400K and ABCG8 DI 9H, and
(c) relating the determined genotype to an assessment of the risk of death due to coronary disease, wherein the presence of one or more genotypes selected from the group consisting of ABCG8 T400K and ABCG8 Y54C indicates an elevated risk of death due to coronary disease.

3. A method according to claim 2, wherein the subject has an elevated serum level of a plant sterol.

4. A method according to claim 3, wherein the plant sterol is selected from sitosterol and campesterol.

5. A method according to claim 4, wherein the plant sterol is sitosterol.

6. A method according to claim 5, wherein the subject is receiving statin therapy.

7. A method according to claim 6, wherein the subject is receiving plant sterol therapy.

8. A method of evaluating a risk of a subject to increased risk of a coronary event, the method comprising the step of determining at least one genotype selected from the group consisting of ABCG8 T400K and ABCG8 D19H as a diagnostic tool for the detection of an increased risk for coronary events in an individual based on an elevated sitosterol concentration in a blood sample from said individual, wherein the risk of a coronary event is increased in a subject having said genotype as compared with a subject without said genotype.

9. A method according to claim 8, wherein the subject has an elevated serum level of a plant sterol.

10. A method according to claim 9, wherein the plant sterol is selected from sitosterol and campesterol.

11. A method according to claim 10, wherein the plant sterol is sitosterol.

12. A method according to claim 10, wherein the subject is receiving statin therapy.

13. A method according to claim 12, wherein the subject is receiving plant sterol therapy.
